(19) United States
(12) Patent Application Publication
(10) Pub. No.: US 2015/0018835 A1
(43) Pub. Date: Jan. 15, 2015

(54) IMPLANT FIXATION MEMBER HOLDER

(71) Applicant: Smith & Nephew, Inc., Memphis, TN (US)

(72) Inventors: Jeffrey J Shea, Memphis, TN (US); Brian W. McKinnon, Arlington, TN (US); Zachary C. Wilkinson, Germantown, TN (US); Michael S Bennett, Southaven, MS (US); Richard D. Lambert, Collierville, TN (US)

(21) Appl. No.: 14/374,593

(22) PCT Filed: Jan. 24, 2013
(86) PCT No.: PCT/US2013/022907

§ 371 Date: Jul. 25, 2014

Related U.S. Application Data
(60) Provisional application No. 61/715,653, filed on Oct. 18, 2012.
(60) Provisional application No. 61/591,057, filed on Jan. 26, 2012.

(51) Int. Cl. A61B 17/17

(52) U.S. Cl. CPC A61B 17/1728
USPC 606/96

(57) ABSTRACT

Systems, devices and methods that improve the placement of orthopedic components and/or instrumentation during surgery. In one particular embodiment, the systems, devices and methods provide drill guides that remove the need for three-handed placement of implant components and reduce complications of implant and fixation member placement. Also disclosed is a sheath with a rounded tip that engages a spherical recess of a patient matched cutting block to reduce the ability of the first fixation pin to communicate unintentional moment/torque to the patient matched instrument through a new articulation between the pin and the guide. Once the first fixation pin has been placed to secure the patient matched instrument to the bone, the effect of subsequent pins is greatly diminished. Subsequent pins may or may not also be decoupled rotationally.

### CROSS-REFERENCE TO RELATED APPLICATIONS

This application claims the benefit of U.S. Provisional Application No. 61/591,057, filed Jan. 26, 2012, and the benefit of U.S. Provisional Application No. 61/715,653, filed Oct. 18, 2012. The disclosure of each prior application is incorporated by reference in its entirety.

### BACKGROUND OF THE INVENTION

1. Field of the Invention

The present invention relates generally to instruments used in orthopedic surgery. More specifically, the invention relates to an initial fixation member drill guide and methods of using the guide.

2. Related Art

During orthopedic surgery, a surgeon must accurately position implant and instrument components, such as acetabular shells, acetabular augments, bone plates, flanges, patient matched cutting guides and the like. Proper orientation of the component is critical for achieving implant stability and fixation, as well as the long-term viability of the implant. For example, acetabular components are typically inserted to achieve desired inclination and anteversion angles while still providing for acetabular rim contact and bone coverage. One way of assuring proper orientation is to hold the component in place with fixation members, such as screws or pins. These members secure the component against the bone either temporarily or permanently. The surgeon usually uses a power drill to place the screws in the bone through openings in the implant or instrument component.

Surgeons have typically held the implant or instrument component and used the other hand to fix the component in place with a pin or screw. Alternatively, they have relied on forceps to hold the implant in place. However, several problems arise with the traditional approaches. Both temporary and permanent fixation members must gain adequate fixation in good quality bone and the fixation member should be accurately centered within the aperture in the implant component. Surgeons have had to use one hand to hold both the implant and the fixation member at the desired locations while using the other hand to operate the power drill. This juggling often results in inadequate positioning of the implant and/or fixation member due to the large moment that can be inadvertently applied through the fixation member and the minimal resistance offered by stabilization by hand, forceps, etc.

An additional complicating factor can be the limited exposure to the implant site. This can make it difficult for a surgeon to put their hands in the wound cavity. For example, the acetabulum of the hip is typically located beneath several inches of body tissue. The need to limit incision size may result in difficulty accessing the implant site and tissue can be bruised or damaged by excessive hand contact.

There is also a need for improvement in the temporary fixation of orthopaedic instrumentation. If with standard, reusable instrumentation there is a rod of substantial length and fixation resisting unintentional moment/torque applied by the user through the pin to the guide, then the corresponding moment/torque resistor within the cutting block would be the surfaces that conform to the patient anatomy; however, due to the anatomic shapes of the distal femur and proximal tibia, the resistive moment/torque sustainable by patient matched contact surfaces is at least 10 times less that of the traditional tibial and femoral intramedullary or extra-medullary rod.

It has been observed through various lab tests that when pinning a patient matched instrument to a bone after manual placement, as much as a two degree shift can occur in any rotational alignment degree of freedom (DOF), such as flexion/extension, varus/valgus, internal/external rotation. This is a problem because patient matched instruments are designed to control implant placement within as small a tolerance as +/−two degrees, so a two degree shift can consume as much as half the total allowable error tolerance.

Thus, there is a continuing need for improving the placement of implant components and instrumentation during orthopaedic surgery.

### SUMMARY OF THE INVENTION

The various embodiments of the present invention described below and shown in the Figures describe systems, devices and methods that improve the placement of orthopedic components and/or instrumentation during surgery. In one particular embodiment, the systems, devices and methods provide drill guides that remove the need for three-handed placement of implant components and reduce complications of implant and fixation member placement.

One aspect of the disclosure is a drill guide for an orthopedic procedure comprising a proximal end, a distal end and a connecting portion, wherein the drill guide has a central bore sized to accept a fixation element.

In some embodiments there is provided a teardrop shaped drill guide, a drill guide made of a disposable material and a drill guide with angulation limiting portions disposed about the distal end.

Another aspect of the disclosure discloses a method of securing an orthopedic implant including placing the implant in position on a patient's bone, holding the implant in position while positioning a drill guide onto a surface feature of the implant, angling the drill guide to a desired orientation, and inserting a fixation member through the implant into the patient's bone.

In some embodiments an implant is selected from an acetabular augment, a femoral augment, a tibial augment, a tibial base, a bone plate, and an acetabular shell or flange.

Yet another aspect of the disclosure is reducing the ability of the initial fixation pin to communicate unintentional moment/torque to the patient matched instrument through a new articulation between the pin and the patient matched instrument. This new articulation may be introduced as the addition of a drill or pin guide sheath with a rounded tip that engages a spherical recess of a patient matched cutting block. Alternatively, this new articulation may be expressed as a variable pin aperture shape having a widened pin entrance opening and a narrow pin exit opening in close proximity to the interface between the patient matched guide and the patient anatomy. Once the initial fixation pin has been placed to secure the patient matched instrument to the bone, there is sufficient stability to resist the unintentionally communicated moment/torque during subsequent pining. Subsequent pins may or may not also be “decoupled” rotationally.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The accompanying drawings, which are incorporated in and form a part of the specification, illustrate the embodiments of the present invention and together with the written description serve to explain the principles, characteristics, and features of the invention. In the drawings:

FIG. 1 shows a side view of a drill guide sheath.

FIGS. 2 and 3 show cross-sections of the drill guide sheath shown in FIG. 1.

FIG. 4 shows a side view of a drill guide sheath being positioned within the fixation member openings of an implant.

FIG. 5 shows a perspective view of a fixation member in the embodiment of FIG. 1.

FIG. 6 shows a perspective view of an acetabular augment within an acetabulum.

FIG. 7 is a detail view of FIG. 6, showing an acetabular augment within an acetabulum with the drill guide sheath contacting the augment.

FIGS. 8A-8D show additional embodiments of the present disclosure.

FIG. 9 shows a side perspective view of a patient matched instrument temporarily mounted on a bone.

FIG. 10 is a partial cut-away view of FIG. 9.

FIG. 11 is a side view of FIG. 10 shown without a pin.

FIG. 12 is a side view of FIG. 10 shown with the pin.

FIGS. 13-16 illustrate pinning a patient matched instrument to bone.

FIGS. 17-19 illustrate yet another additional embodiment of the present disclosure.

### DETAILED DESCRIPTION OF THE EMBODIMENTS

The following description of the preferred embodiment(s) is merely exemplary in nature and is in no way intended to limit the invention, its application, or uses.

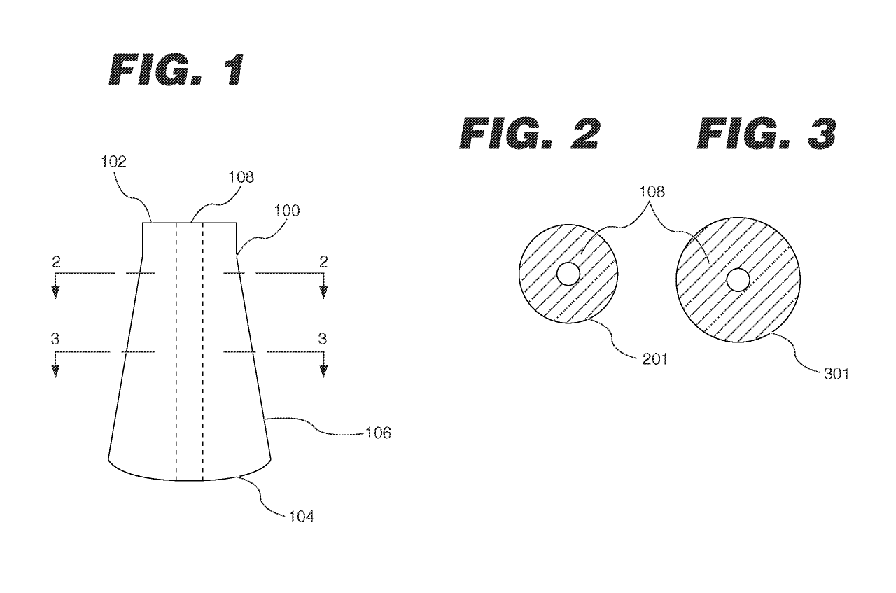

FIG. 1 illustrates a drill guide sheath 100. The drill guide sheath also may be called a bulb. Drill guide sheath 100 has a proximal end 102 and a distal end 104, connected by an exterior surface 106. FIGS. 2 and 3 show circular cross sections 201 and 301 of drill guide sheath 100, but the exterior surface 106 also may have ridges, indentations or other surface features to allow a user to more easily manipulate it into position over a fixation member. Passageway 108 extends from proximal end 102 to distal end 104 and is sized and shaped to allow passage of a fixation member, such as a speed pin, bone screw, bone pin, or Kirschner wire.

In FIG. 1, distal end 104 is shaped to allow the user to angle the fixation member within a range of angles when the drill guide sheath 100 is mounted on openings on an implant or instrument. As examples, distal end 104 may be ellipsoidal or spherical. In certain embodiments, the geometry of the drill guide sheath may vary to allow a different range of angles for the fixation member. The exterior surface 106 may be radiused adjacent to the distal end 104. In the depicted embodiment, the exterior surface 106 has a first portion adjacent the proximal end that is generally cylindrical and a second portion that is generally conical. In some embodiments, the exterior surface is tear-dropped shaped. In yet other embodiments, the entire exterior surface 106 is conical. In general, the distal end is configured to interface with an opening of an implant or instrument to maintain an on-center position of the opening.

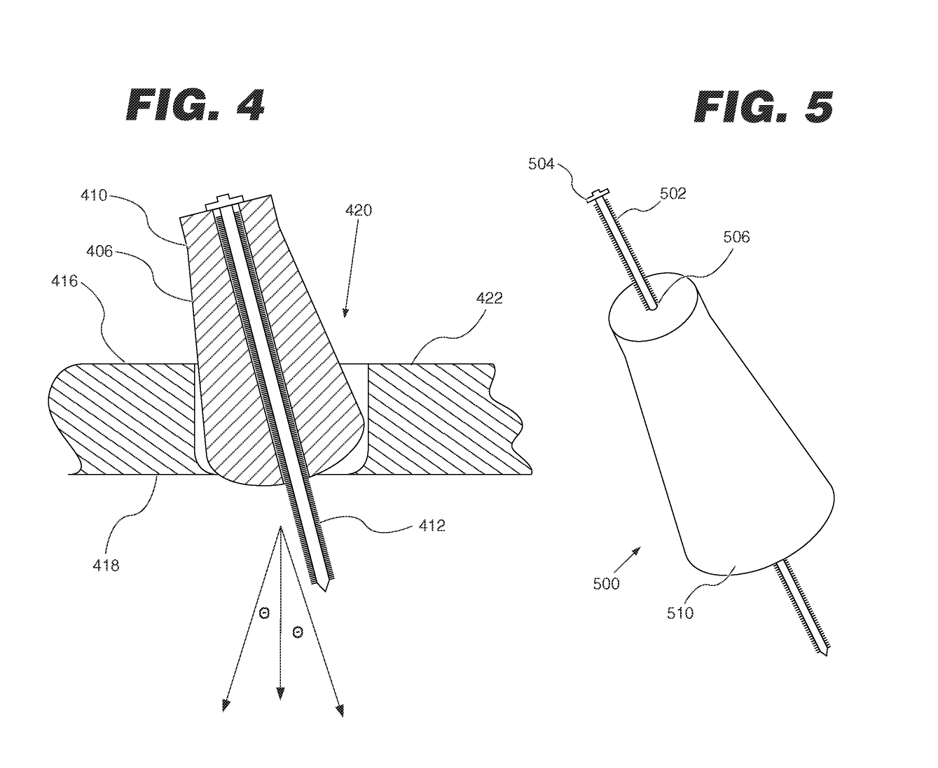

FIG. 4 depicts a cross-section of drill guide sheath 410 positioned relative to implant 422. Implant 422 is provided with top and bottom layers 416 and 418, respectively. In the depicted embodiment, fixation member opening 420 is an opening extending through implant 422, but the present invention is also applicable to blind holes. When the drill guide sheath 410 is placed on the fixation member 412 and positioned within the fixation member opening 420, the fixation member 412 may be adjusted about any angle Θ, allowing the user to place fixation member 412 at a variety of angles. In this instance, Θ is limited by exterior surface 406 of the drill guide sheath 410 contacting the top layer 416 and/or a wall of the opening 420 of the implant 422. In one embodiment, Θ has a range of zero to about thirty degrees. In one particular embodiment, Θ has a range of zero to about fifteen degrees.

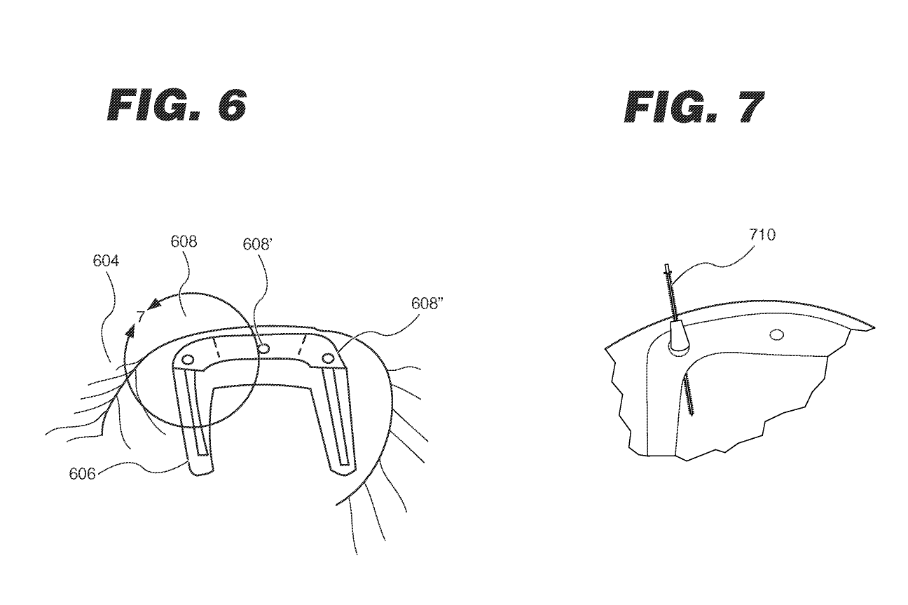

FIGS. 5-7 depict other aspects of using the drill guide sheath of the present disclosure. In FIG. 5, fixation member 502 is inserted through the opening 506 in drill guide sheath 500. Fixation member 502 may have an extended surface 504 disposed about the periphery of the proximal end of 502 that serves to limit how long fixation member 502 extends from distal end 510 of the drill guide sheath 500. Extended surface 504 serves also to limit the depth a fixation member can ultimately be implanted into a patient's bone. Fixation members may be supplied in a variety of lengths and diameters.

FIG. 6 illustrates a patient's exposed acetabulum 604 with augment 606 placed therein. Implant fixation openings 608, 608′ and 608″ are provided in augment 606. Implant fixation openings 608, 608′ and 608″ may look similar to opening 420 depicted in FIG. 4. As a preliminary step in securing augment 606, a user, such as a surgeon, may attach the drill guide sheath 500 of FIG. 5 onto the end of a drill and position distal end 510 into opening 608. Securing augment 606 with one hand, the user can then easily angle fixation member 710 and drill with the other hand. Because the drill guide sheath 500 is resting on opening 608, distal end 510 of the drill guide sheath 500 allows for fixation member angles to be easily changed. In some embodiments, a portion of the opening may be concave to receive the convex shape of the distal end of the drill guide sheath. When the user has the angle they desire, they can simply set fixation member 710 in place, either manually or with the assistance of a power drill. As previously noted, depth of a fixation member in bone can be controlled by extended surface 504.

With the implant provisionally in place, this frees up a hand for the user to then place additional fixation members through openings 608′ and/or 608″. Finally, the user can remove drill guide sheath 500 and then reinsert a permanent fixation member (such as a screw or pin) into opening 608. The two main advantages are that the user can focus their efforts on positioning augment 606 in the proper location and positioning fixation members in the proper orientation.

The drill guide sheath can be made of any number of materials, including polymers and metals. One preferred polymer is polyether ether ketone (PEEK) but the material used for the guide is not critical. Some exemplary characteristics are an inexpensive material that will not shed excessive particles in use and is capable of sterilization. In some embodiments, the drill guide sheath is made of a radiopaque material.

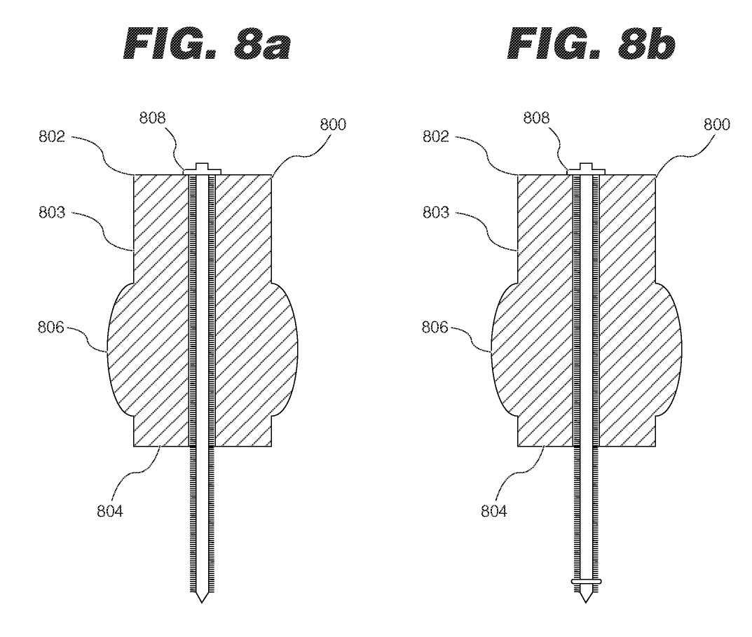

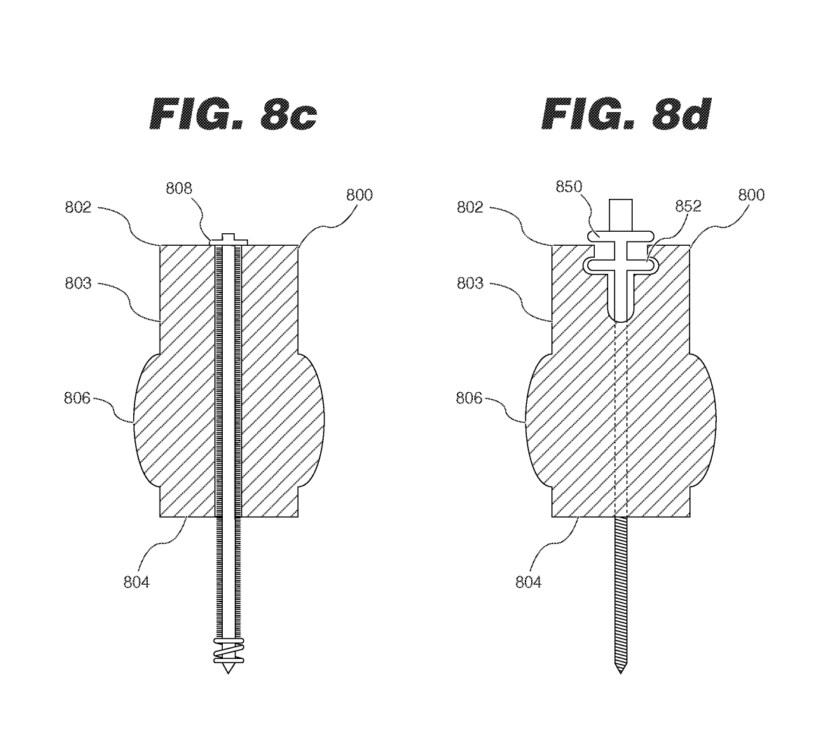

FIG. 8A illustrates yet another embodiment of the present disclosure. FIG. 8A is similar to the embodiment shown in FIG. 1, except for protrusion 806 on the exterior surface 803 of drill guide sheath 800. The protrusion also may be called a bulge. In the depicted embodiment, the exterior surface 803 is generally cylindrical but could also be conical or part-cylindrical/part-conical. Protrusion 806 serves to contact side walls of an opening in an implant or instrument. In some embodiments, the fixation member may have a locking feature to prevent the fixation member from separating from the drill guide sheath. In FIG. 8B, the locking feature is illustrated as an enlarged portion near the distal end of the fixation member but other mechanisms may equally be used. In FIG. 8C, the locking feature is shown as section of threads that have a mean diameter larger than the diameter of the threads elsewhere on the shaft of the fastener. The portion with the enlarged diameter may or may not have the same pitch as the rest of the fixation member. In FIG. 8D, the locking feature is an engagement member that snaps into a receptacle of the drill guide sheath. In the depicted embodiment, the engagement member is an annular flange 852 that mates with an annular groove of the drill guide sheath. In some embodiments, the drill guide sheath may have a first flange 850 to place the fixation member relative to a collet (not shown) and the annular flange 852 to catch and hold the drill guide sheath when it is fully seated.

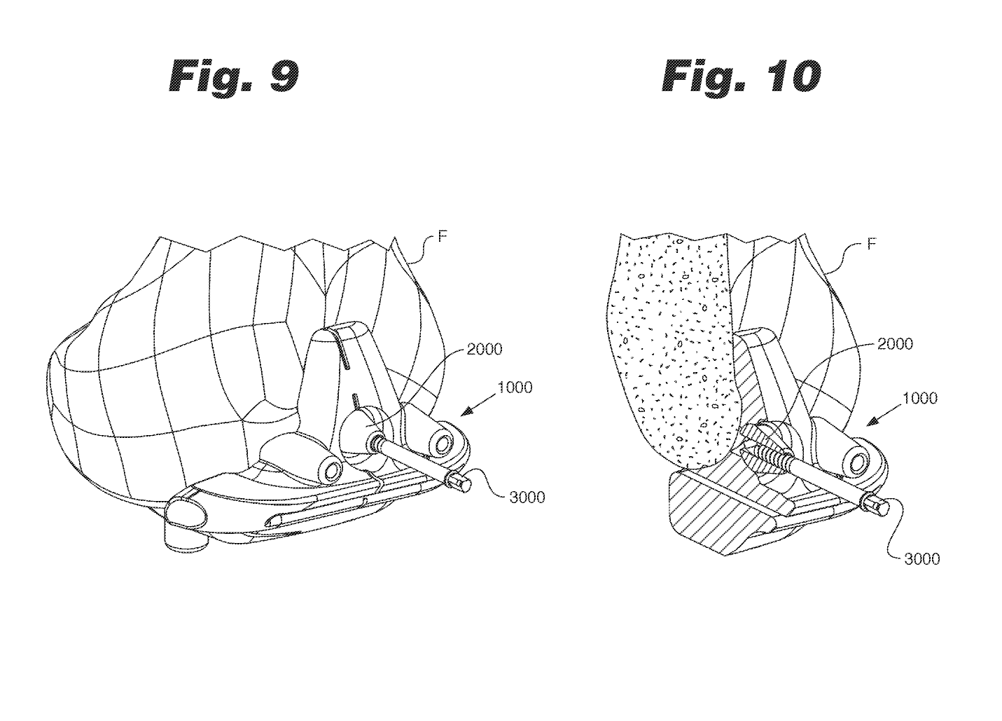

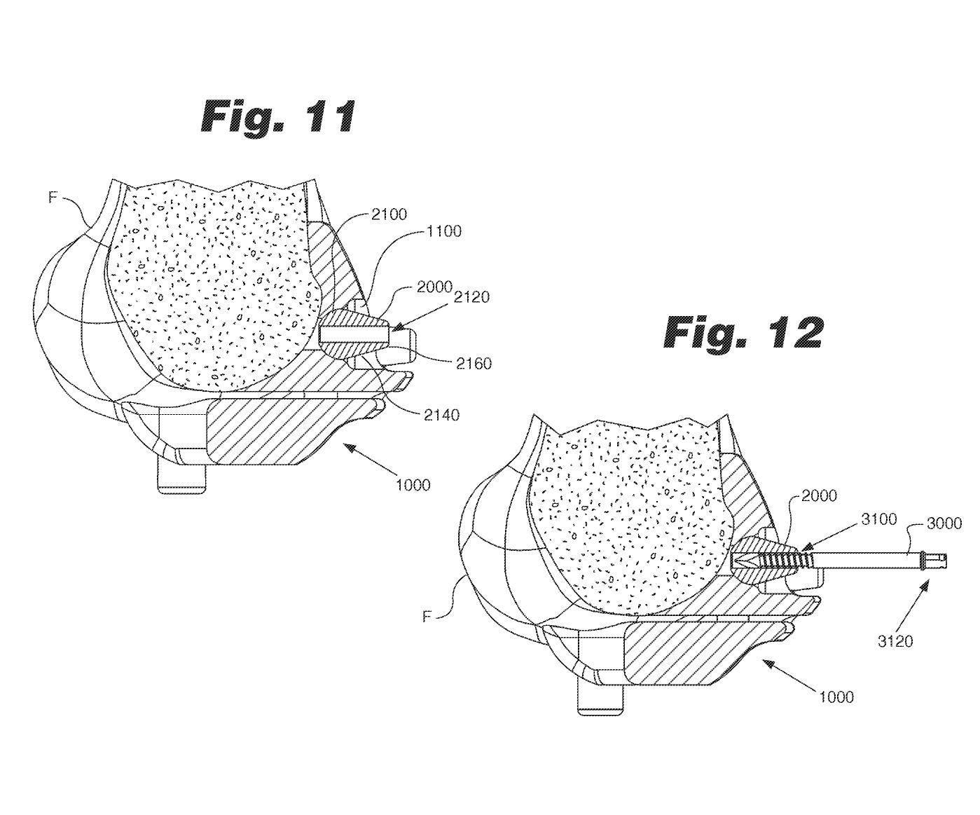

FIGS. 9-12 show a patient matched instrument 1000 temporarily mounted on a bone, such as a femur F. The patient matched instrument has at least one generally concave recess 1100. A sheath 2000 is adapted to engage the generally concave recess 1100. The sheath 2000 has a convex tip 2100 and a longitudinal opening 2120. An outer surface 2140 of the sheath may be tear-dropped in shape or cylindrical. The longitudinal opening is sized and shaped to receive a pin 3000. The pin 3000 may include threads 3100. The pin 3000 may have a head 3120. The pin 3000 is inserted into the sheath 2000, through the patient matched instrument 1000, and into the bone. The head 3120 of the pin engages a shoulder 2160 of the sheath 2000 to compress the sheath against the patient matched instrument 1000. Because the pin 3000 imparts a moment or torque only to the sheath 2000, and not the patient matched instrument 1000, greater accuracy in placing the patient matched instrument is achieved.

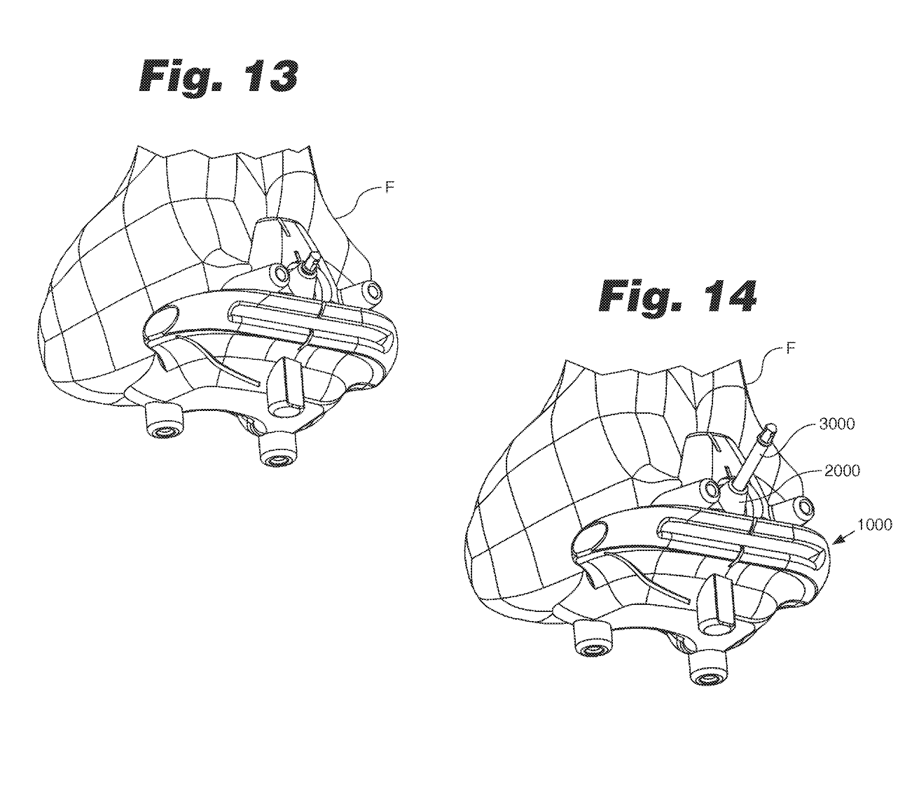

In FIG. 13, the sheath 2000 and the pin 3000 are mated to the patient matched instrument 1000 in a first particular orientation. In FIG. 14, the sheath and pin are moved to a second orientation without imparting an angular error to the patient matched instrument 1000.

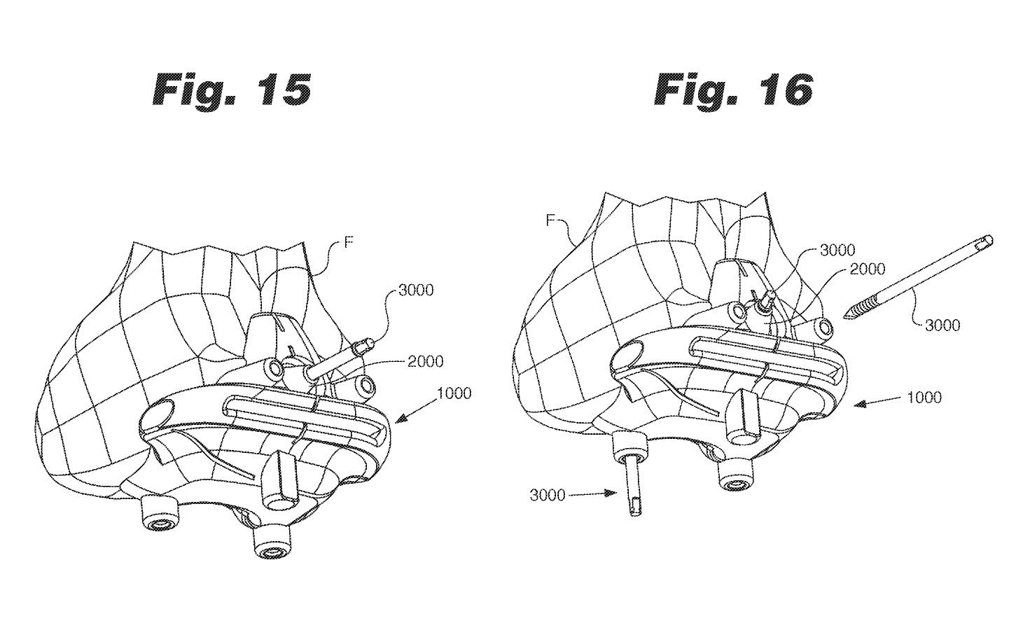

In FIG. 15, the headed pin 3000 is seated on the sheath 2000, and the sheath 2000 is seated on the patient matched instrument 1000. In FIG. 16, additional fixation pins 3000 are inserted through the patient matched instrument 1000 and into bone. Because the first fixation pin 3000 does not impart an angular moment and temporarily secures the patient matched instrument, the additional fixation pins are less likely to impart an error into the placement of the patient matched instrument. While it is significant to rotationally decouple the first fixation pin, subsequent fixation pins do not necessarily need to be decoupled.

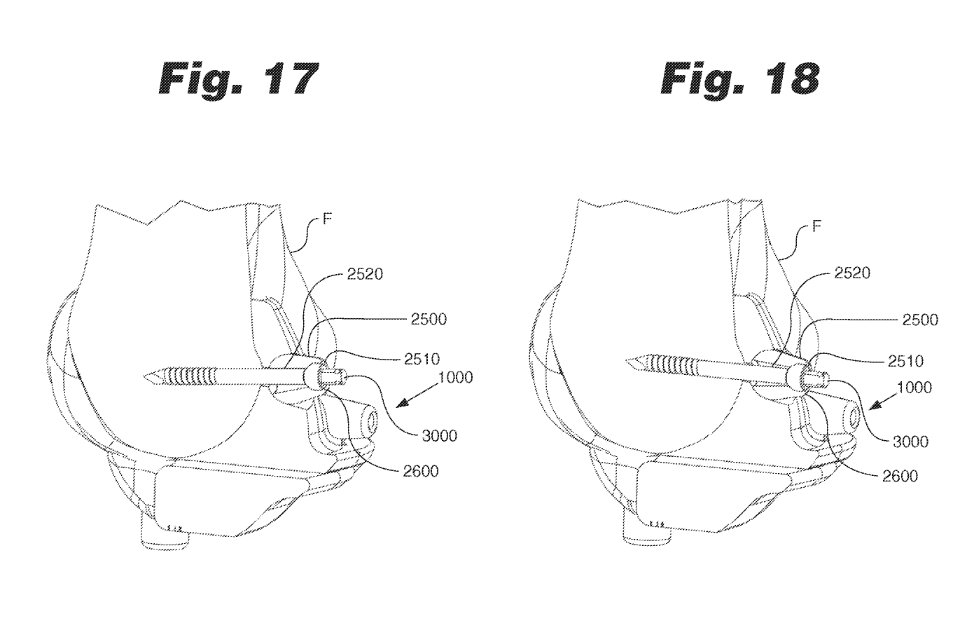

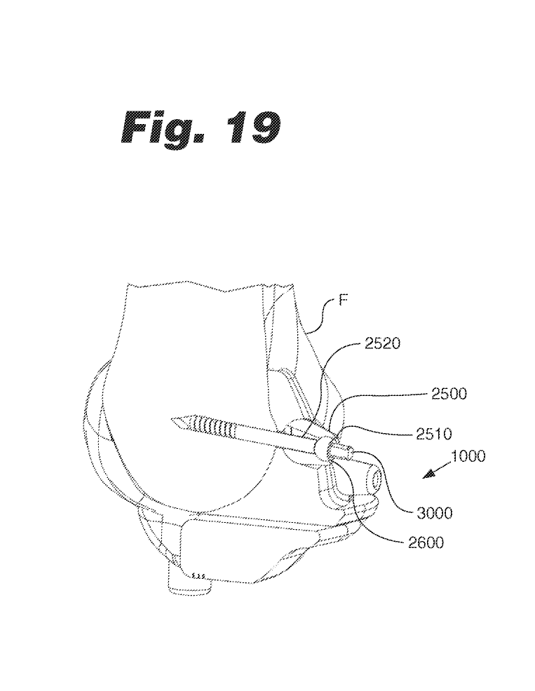

FIGS. 17-19 illustrate yet another additional embodiment of the present disclosure. In FIGS. 17-19 the sheath 2500 has a conical internal opening 2520 and a seat 2510 on the proximal end. In some embodiments, the seat 2510 may be concave. A cup 2600 mates with the fixation member 3000 and sits within seat 2510. The cup 2600, the seat 2510, and the conical internal opening 2520 allow the fixation member to pivot relative to the sheath 2500. In other words, the proximal end of the sheath 2500 serves as the origin or vertex for allowed angular variation between the fixation member and sheath Thus, the fixation member 3000 can pivot relative to the sheath 2500, and the sheath 2500 is free to pivot relative to the instrument 1000. The cup 2600 may be made from the same material as the sheath 2500 or a different material. The cup 2600 may be spherical or ellipsoidal. The cup 2600 may include a locking or snap feature to lock onto the fixation member 3000.

There are many variations of articular surfaces between the fixation member and sheath or the sheath and patient matched instrument (PMI). All of them allow for between two and forty degrees of relative movement between the PMI and the fixation member in all DOF. In some embodiments, relative movement is between five and thirty degrees. In other embodiments, the relative movement is between five and fifteen degrees.

Another embodiment includes a specialized connection between the pin driver and the fixation member. The connection includes an articulation that does not communicate moment/torque from the driver to the fixation member, and the fixation member is free to be constrained by the PMI rather than the driver.

A method of implanting an augment is also disclosed. The method includes the steps of: providing an augment with at least one opening; providing a drill guide sheath having a proximal end, a distal end and an exterior surface, a central bore sized and shaped to accept a fixation element connects the proximal end and the distal end, and the distal end is convex in shape; inserting a fixation member into the central bore; placing the convex distal end of the drill guide sheath into the at least one opening; and affixing the fixation member into bone. In some embodiments, the method includes the step of placing an additional fixation member into at least one other opening. In some embodiments, the method includes the steps of removing the fixation member, removing the drill guide sheath, and placing a permanent fixation member into bone.

A method of temporarily fixating a patient matched cutting block is also disclosed. The method includes the steps of: providing a patient matched instrument with at least one opening; providing a drill guide sheath having a proximal end, a distal end and an exterior surface, a central bore sized and shaped to accept a fixation element connects the proximal end and the distal end, and the distal end is convex in shape; inserting a fixation member into the central bore; placing the patient matched instrument on bone; placing the convex distal end of the drill guide sheath into the at least one opening; and affixing the fixation member into bone.

As various modifications could be made to the exemplary embodiments, as described above with reference to the corresponding illustrations, without departing from the scope of the invention, it is intended that all matter contained in the foregoing description and shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

What is claimed is:

1. A drill guide sheath for an orthopedic procedure comprising:
a proximal end, a distal end spaced apart from the proximal end, and an exterior surface, a central bore sized and shaped to accept a fixation member, the central bore connecting the proximal end and the distal end, the distal end is convex in shape, and the exterior surface is selected from the group consisting of tear-dropped shape, conical, and a first portion that is cylindrical and a second portion that is conical.

2. The drill guide sheath according to claim 1, wherein the distal end is configured to interface with an opening of an implant or instrument to maintain an on-center position of the opening.

3. The drill guide sheath according to claim 1, wherein the distal end has a shape selected from the group consisting of ellipsoidal and spherical.

4. The drill guide sheath according to claim 1, wherein the exterior surface further comprises a bulge that serves to contact side walls of an opening in an implant or instrument.

5. The drill guide sheath according to claim 1, further comprising a shoulder on the proximal end.

6. The drill guide sheath according to claim 1, wherein the exterior surface is radiused adjacent the distal end.

7. The drill guide sheath according to claim 1, wherein the exterior surface is tear-dropped shape.

8. The drill guide sheath according to claim 1, wherein the exterior surface is conical.

9. The drill guide sheath according to claim 1, wherein the exterior surface has a first portion that is cylindrical and a second portion that is conical.

10. The drill guide sheath according to claim 1, wherein the bore is conical to allow angular variation between the axis of the sheath and the axis of the fixation member.

11. The drill guide sheath according to claim 10, wherein the proximal end is shaped such it serves as the origin or vertex for allowed angular variation between the fixation member and the sheath.

12. A drill guide sheath for an orthopedic procedure comprising:
a proximal end, a distal end spaced apart from the proximal end, and an exterior surface, a central bore sized and shaped to accept a fixation member, the central bore connecting the proximal end and the distal end, the distal end is convex in shape, the exterior surface is conical, and the distal end has a shape selected from the group consisting of ellipsoidal and spherical.

13. The drill guide sheath according to claim 12, wherein the distal end is configured to interface with an opening of an implant or instrument to maintain an on-center position of the opening.

14. The drill guide sheath according to claim 12, wherein the exterior surface further comprises a bulge that serves to contact side walls of an opening in an implant or instrument.

15. The drill guide sheath according to claim 12, further comprising a shoulder on the proximal end.

16. The drill guide sheath according to claim 12, wherein the exterior surface is radiused adjacent the distal end.

17. The drill guide sheath according to claim 12, wherein the exterior surface is tear-dropped shape.

18. The drill guide sheath according to claim 12, wherein the exterior surface has a first portion that is cylindrical and a second portion that is conical.

19. The drill guide sheath according to claim 12, wherein the bore is conical to allow angular variation between the axis of the sheath and the axis of the fixation member.

20. A drill guide sheath for an orthopedic procedure comprising:
a proximal end, a distal end spaced apart from the proximal end, and an exterior surface, a central bore sized and shaped to accept a fixation member, the central bore connecting the proximal end and the distal end, the distal end is spherical and convex in shape, and the exterior surface is selected from the group consisting of tear-dropped shape, conical, and a first portion that is cylindrical and a second portion that is conical.
